(19) United States
(12) Patent Application Publication
(10) Pub. No.: US 2010/0324530 A1
(43) Pub. Date: Dec. 23, 2010

(54) HOLLOW NEEDLE

(76) Inventor: Bernard Hertzog, Paris (FR)

Correspondence Address: Osler, Hoskin & Harcourt LLP (JurisPatent), 1000 de La Gauchetiere Street West, Suite 2100, Montreal, QC, H3B 4W5, CA

(21) Appl. No.: 12/865,560

(22) PCT Filed: Feb. 3, 2009
(86) PCT No.: PCT/EP09/00698

§ 371 Date: Jul. 30, 2010

Related U.S. Application Data
(60) Provisional application No. 61/066,501, filed on Feb. 21, 2008.

(30) Foreign Application Priority Data
Feb. 4, 2008 (FR) 0800583
Mar. 28, 2008 (EP) 08103053.8

(51) Int. Cl. A61M 5/32

(52) U.S. Cl. 604/506; 604/239

(57) ABSTRACT

The present invention is a hollow needle for attachment to a syringe, through which a liquid can be delivered to the stratum granulosum and/or beneath the epidermis, preferably to an area consisting from the stratum granulosum (included) to reticular dermis (included) and more preferably beneath the papillary dermis during a cosmetic procedure, the needle comprising a distal tip and including a lateral aperture adjacent to the distal tip, wherein the liquid exits the needle via the lateral aperture, and wherein the needle is flexible. Apparatus comprising a needle of the invention and a syringe, and a method of use thereof, is also provided.

### CROSS-REFERENCE

The present application is the U. S. National Stage of PCT/EP2009/000698 filed Feb. 3, 2009. The present application claims the benefit of priority to French patent application no. 08 00583 filed Feb. 4, 2008, United States Provisional Application No. 61/066,501, filed Feb. 21, 2008, and European Patent Application No. 08.103 053.8, filed Mar. 28, 2008. All of these applications are incorporated herein by reference.

### FIELD OF THE INVENTION

The present invention relates to needles for delivering a liquid to the stratum granulosum and/or beneath the epidermis, preferably to an area consisting from the stratum granulosum (included) to reticular dermis (included) and more preferably beneath the papillary dermis during an injection procedure and more preferably during a cosmetic procedure.

### BACKGROUND OF THE INVENTION

Many cosmetic procedures carried out on the skin involve the injection of a liquid beneath the epidermis. An example of such a liquid is hyaluronic acid. For injection beneath the epidermis, the needle may pass through the stratum papillaire. The stratum papillaire contains a network of blood vessels that, if severed, may lead to facial bruising.

Injections of a liquid into the stratum granulosum and/or beneath the epidermis during a cosmetic procedure are traditionally carried out using a conventional sharp pointed needle. One example of such a needle is a 25 G needle, which has a diameter of 0.5 mm. Due to its sharp tip, the depth of penetration is difficult to control, and the needle can cut the deep-lying tissues of the skin. In particular, the needle may sever the cellular tissues, the elastic and collagen fibers, and the fine blood and lymph vessels. As the depth of penetration is difficult to control, the administration may not be precise, and the cosmetic procedure may therefore be less effective than desired.

A cosmetic treatment to the skin may require several injections of a liquid. Before each injection with a conventional sharp-tipped needle, anaesthesia is required. This may require a block over a large area.

### SUMMARY OF THE INVENTION

The present invention is based on the discovery that, by using a flexible needle with a blunt tip, liquid can be delivered over a large area of the stratum granulosum and/or beneath the epidermis, preferably to an area consisting from the stratum granulosum (included) to reticular dermis (included) and more preferably beneath the papillary dermis, through one micro-incision or preferably through a tiny hole previously made with a classic needle. This means that the amount of anaesthesia needed before a cosmetic procedure may be reduced by 10 to 15-fold. As the needle is blunt and flexible, the risk of severing the blood vessels underneath the epidermis may also be reduced and the risk of intravascular injection is abolished. The combination of this advantages leads to a shortening of the cosmetic procedure and of the period of recovery.

According to a first aspect, the present invention is a hollow needle for attachment to a syringe, through which a liquid can be delivered to the stratum granulosum and/or beneath the epidermis, preferably to an area consisting from the stratum granulosum (included) to reticular dermis (included) and more preferably beneath the papillary dermis during a cosmetic procedure, the needle comprising a distal tip and including a lateral aperture adjacent to the distal tip, wherein the liquid exits the needle via the lateral aperture, and wherein the needle is flexible.

It is know from application US2001/003709 a rigid and curved canulae for injections into muscle comprising a distal tip and including a lateral aperture adjacent to the distal tip. These canulaes have a diameter of 1.5 mm. They are totally different from the needle according to the invention which is flexible, preferably straight and has preferably a far smaller diameter. Furthermore the needle according to the invention allows preferably the injection of a liquid to an area consisting from the stratum granulosum (included) to reticular dermis (included)) whereas the canulae according to US2001/003709 is strictly dedicated to the injection into muscles.

According to a second aspect, the present invention is an apparatus for delivering a liquid to the stratum granulosum and/or beneath the epidermis, preferably to an area consisting from the stratum granulosum (included) to reticular dermis (included) and more preferably beneath the papillary dermis during a cosmetic procedure, comprising a syringe and a needle, as defined above.

According to a preferred embodiment, the needle according to the invention is straight.

According to a third aspect, the present invention is a method of delivering a liquid to the stratum granulosum and/or beneath the epidermis, preferably to an area consisting from the stratum granulosum (included) to reticular dermis (included) and more preferably beneath the papillary dermis during a cosmetic procedure, using apparatus, as defined above.

According to a fourth aspect, the present invention is a method of treatment comprising the use of a method of delivering a liquid as defined above.

### DESCRIPTION OF PREFERRED EMBODIMENTS

The invention will now be illustrated by way of example only with reference to the following drawings.

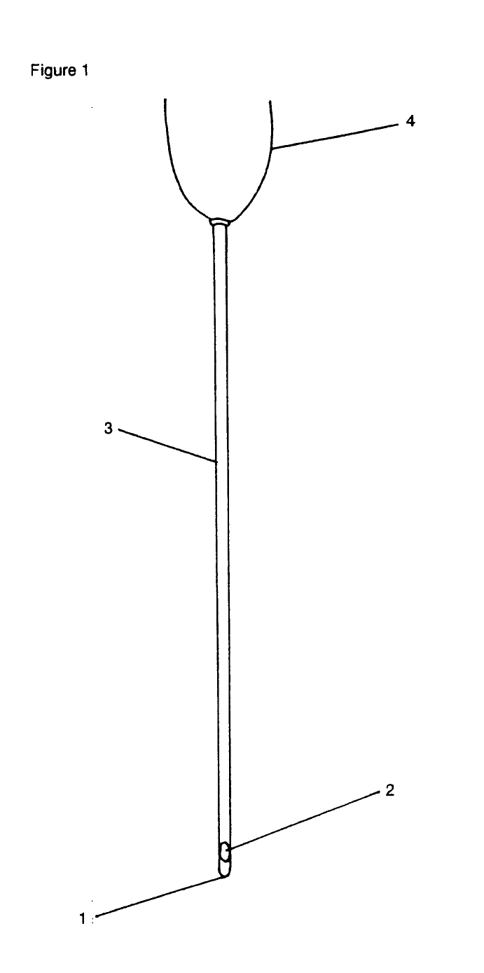

FIG. 1 shows a schematic lateral section of a needle embodying the present invention. The needle (3) includes a hollow base section (4), which may be made of a plastics material. This base section (4) allows the needle to be attached to a syringe containing a liquid for subepidermal administration. The needle (3) has a lateral aperture (2), through which the liquid is delivered. The distal tip (1) of the needle is blunt.

The length of a needle according to the invention is preferably between 13 and 45 mm. In another preferred embodiment, the length of the needle is less than 50 mm, more preferably between 5 and 50 mm. In a further preferred embodiment, the length of the needle is approximately 47, 40 mm, 37, 27, 20,17, 13 and 10.

A needle according to the invention preferably has a diameter of between 0.3 and 0.7 mm. More preferably, the diameter is less 0.5, 0.4 or 0.3 mm. More preferably still, the needle has a diameter of approximately 0.5 mm, 0.4 mm, 0.3 mm, 0.2 mm, between 0.2 and 0.3 mm, between 0.3 and 0.4 mm. or between 0.4 and 0.5 mm.

The fine diameter of the needle may help to reduce the risk of severing the blood vessels.

Due to the lateral aperture, the tip of the needle is blunt. Preferably, the tip of needle is rounded. This may reduce the trauma to the patient. The lateral length of the aperture is preferably approximately equal to 0.5 mm. In a preferred embodiment, the aperture is located approximately 1 mm from the distal tip of the needle. According to another preferred embodiment, the aperture is located at most at 2 mm from the distal tip of the needle and even more preferably between 0.05 mm and 2 mm from the distal tip of the needle and according to a definitely preferred embodiment between 0.05 mm and 0.5 mm from the distal tip of the needle.

According to a preffered embodiment, the needle according to the invention has only one lateral aperture and has no aperture at its distal tip.

The size of the larger diameter of the lateral aperture is preferably comprised between 0.01 and 1 mm more preferably between 0.01 and 0.2 mm, even more preferably between 0.01 and 0.1 mm. The one skilled in the art is able to determine the most accurate size in regard of the viscosity for each liquid to be injected and of the specific therapeutic use.

According to a preferred embodiment, the hollow base section of the needle according to the invention further comprises a sign (e.g. a color spot) and preferably at the same radial position than the lateral aperture. This sign allows knowing the position of the lateral aperture even when the needle is in position under the skin of a patient.

A needle of the invention is flexible, meaning that it may be able to glide through the layers of the skin without damaging the underlying tissues, or cutting the blood vessels. This may help to reduce the amount of bruising after a particular cosmetic procedure. According to a more preferred embodiment, the term “flexible” means that the needle according to the invention can be bend to an angle of 60° relative to its longitudinal axis, by applying a force to the distal tips which is less than 5N.

The needle according to the invention can be made totally or partially in stainless steel. According to another embodiment of the invention can be totally or partially made of a shape memory alloy (SMA). The use of such SMA allows producing a needle which can return to a specific shape when it is submitted to the body temperature. According to a preferred embodiment, the needle of the invention is partially or totally made of nitinol.

Due to the flexibility of a needle of the invention, and the resistance offered by the deeper layers of the skin, a liquid can be delivered to the stratum granulosum over a large area, and in an even manner, through one single micro-incision of the skin. The tension of the deep-lying layers of skin prevents the blunt needle from penetrating into those layers, and causes it to “curve” into the stratum granulosum and/or beneath the epidermis, preferably to an area consisting from the stratum granulosum (included) to reticular dermis (included) and more preferably beneath the papillary dermis.

The user may be able to manipulate the needle in a “circular” motion, in order to maximize the area of delivery of the liquid. Due to the construction of the needle, it can glide under the skin with ease. This may significantly reduce the pain and trauma to the patient.

Further, as a needle of the invention is able to deliver a liquid to a large area, through one single initial micro-incision, a dosage of anaesthetic to that single micro-incision site may provide sufficient anaesthesia for the injection of all the liquid required for the cosmetic procedure. If a conventional sharp-tipped, inflexible needle were used, several micro-incisions, and several dosages of anaesthetic would normally be required.

By way of example, a cosmetic treatment using conventional sharp-tipped needles may require a dosage of anaesthetic at 20 different sites followed by 20 injections of a liquid such as hyaluronic acid, into each those sites. The same procedure using a needle of the invention may require anaesthetic to be administered to a single site only, followed by a single injection of hyaluronic through one single micro-incision. Due to the construction of the needle, the same area can be treated, as if the procedure was carried utilising the conventional method. The requirement far one micro-incision only may greatly reduce the negative side-effects of the cosmetic procedure, such as bruising and scarring. The procedure may also be more precise, leading to more aesthetically pleasing results.

In one embodiment, a kit is provided, comprising a needle of the invention and a syringe. The syringe may contain the liquid medicament, an example al which is hyaluronic acid. Alternatively, the medicament may be provided separately. In another embodiment, the needle of the invention and the syringe may be integral.

According to another embodiment, the present invention is a method of delivering a liquid to the stratum granulosum and/or beneath the epidermis, preferably to an area consisting from the stratum granulosum (included) to reticular dermis (included) and more preferably beneath the papillary dermis, using apparatus, as defined above. According to a preferred embodiment, the liquid to be delivered is a filler liquid and more preferably is hyaluronic acid. According to another preferred embodiment the liquid to be delivered is a anaesthetic and more preferably is lidocaïn.

According to another embodiment, the present invention is a method of treatment comprising the use of a method of delivering a liquid as defined above. According to a preferred embodiment, the treatment is a cosmetic procedure or anaesthesia.

The following Example illustrates the invention.

### Example

After disinfecting the skin, a small quantity Of anaesthetic is injected (10 to 15 times less than the usual amount when using conventional needles). One single micro-incision is then created using a conventional 25 G or 26 G needle. This micro-incision should be placed in the centre of the area to be treated. The entire length of a needle according to the invention is then inserted laterally into the stratum granulosum through this micro-incision, and the desired liquid (for example, reticulated hyaluronic acid) is injected beneath the skin. The injection is then repeated in a radial pattern, resembling the “spokes-of-a-wheel”, always starting from the same initial micro-incision. Bruising is greatly reduced compared with a conventional procedure, using sharp-tipped needles.

1. A hollow needle for attachment to a syringe, through which a liquid can be delivered to at least one of the stratum granulosum and beneath the epidermis during a cosmetic procedure, the needle comprising a distal tip and including a lateral aperture adjacent to the distal tip, wherein the liquid exits the needle via the lateral aperture, wherein the needle is straight, flexible and wherein the distal tip of the needle is blunt and rounded and wherein a diameter of the needle is less than 0.5 mm.

2. A needle according to claim 1, wherein the diameter of the needle is one selected from the group consisting of about 0.5 mm, about 0.4 mm, about 0.3 mm, about 0.2 mm, between about 0.3 and about 0.4 mm, between about 0.4 mm and about 0.5 mm, and between about 0.2 and about 0.3 mm.

3. A needle according to claim 1, wherein a length of the needle is not greater than 50 mm.

4. A needle according to claim 3, wherein the length of the needle is between 5 mm and 50 mm.

5. A needle according to claim 1, wherein a length of the needle is one selected from the group consisting of 46 mm, 40 mm, 37 mm, 32 mm, 20 mm, 17 mm, and 13 mm.

6. A needle according to claim 1, wherein a size of a larger diameter of the lateral aperture is between 0.05 and 1 mm.

7. A needle according to claim 1, wherein the lateral aperture is located at most at 2 mm from the distal tip of the needle.

8. A needle according claim 1, wherein the needle is at least partially made of one of stainless steel and a shape memory alloy.

9. An apparatus for delivering a liquid to at least one of the stratum granulosum and beneath the epidermis, during a cosmetic procedure, comprising a syringe and a needle according to claim 1.

10. A method of delivering a liquid to at least one of the stratum granulosum and beneath the epidermis, during a cosmetic procedure, comprising using the apparatus according to claim 9.

11. A method according to claim 10, wherein the cosmetic procedure is carried out on the face.

12. A method of delivering a liquid to at least one of the stratum granulosum and beneath the epidermis, comprising using a needle according to claim 1.

13. A method of treatment comprising the use of a method of delivering a liquid according to claim 12.

14. A needle according to claim 1, through which a liquid can be delivered to an area between the stratum granulosum and the reticular dermis.

15. A needle according to claim 1, through which a liquid can be delivered beneath the papillary dermis.

16. An apparatus for delivering a liquid to at least one of the stratum granulosum and beneath the epidermis during a cosmetic procedure, comprising a syringe and a needle according to claim 2.

17. An apparatus for delivering a liquid to at least one of the stratum granulosum and beneath the epidermis during a cosmetic procedure, comprising a syringe and a needle according to claim 3.

18. A method of delivering a liquid to at least one of the stratum granulosum and beneath the epidermis comprising using a needle according to claim 2.

19. A method of delivering a liquid to at least one of the stratum granulosum and beneath the epidermis comprising using a needle according to claim 3.

20. A method of delivering a liquid to at least one of the stratum granulosum and beneath the epidermis comprising using a needle according to claim 6.
